(19) United States
(12) Patent Application Publication
(10) Pub. No.: US 2007/0203392 A1
(43) Pub. Date: Aug. 30, 2007

(54) Massage device

(76) Inventor: Ivar Jerstad, Oslo (NO)

Correspondence Address: JAMES E. BRUNTON, ESQ., P. O. BOX 29000, GLENDALE, CA, 91209, US

(21) Appl. No.: 11/363,829

(22) Filed: Feb. 28, 2006

(51) Int. Cl. A61F 5/00

(52) U.S. Cl. 600/38

(57) ABSTRACT

A massage device that can be strategically positioned between the user's legs in such a manner that upon operation of the hand-grip component of the device, the massage head of the device will be moved upwardly and downwardly relative to the user's penile head so as to cause intense pleasure to the user. Advantageously, a plurality of massage heads of different shapes can be readily attached to the longitudinally extending support of the device to which the hand-grip component is pivotally connected.

### BACKGROUND OF THE INVENTION

1. Field of the Invention

The present invention relates generally to massage devices. More particularly the invention concerns a massage device for pleasurably massaging the user at a location proximate the penile root.

2. Discussion of the Prior Art

Considerable effort has been undertaken in the past to determine the characteristics of a natural erection in the human male. This research has determined that that an erection is quite complex and involves multiple factors, including psychic, hormonal, neurological and vascular. However, when viewed merely relative to its mechanical aspect, it is well understood that when blood flows with sufficient pressure into the cavernous bodies of the penis, these bodies become swollen with the blood being sufficiently retained therein by a valvular system which impairs the free return of the blood to the organism's general circulating system.

A number of approaches have been taken in the past to develop mechanical devices that will increase blood flow toward the penis and provide sexual pleasure for the male user. One such early device is described in U.S. Pat. No. 580,553 issued to Laib. This patent relates to a massaging apparatus for medicinal-therapeutical treatment of human genitals. Basically the device comprises a carrier tube that is enclosed in a toroidal rubber hose that is filled with an anti-friction mass like glycerin. Pairs of support rollers inside of the hose are journalized on the tube in encircling relationship thereof. Driving rollers, each associated to one pair of support rollers, are in engagement with the rubber hose supported by the inner support rollers for effecting revolving movement of the hose longitudinally around the tube. The drive rollers encircling the hose on the tube are rotatably supported in a common frame or housing and by their engagement with the support rollers at the same time maintain and support the carrier tube and hose.

Another early device is described in U.S. Pat. No. 3,910,262 issued to Stoughton. The Stoughton patent discloses a therapeutic apparatus intended for producing orgasms in males and females. It contains a motor driven hydraulic actuator which produced positive and negative pneumatic pressure pulses. These pressure reversals produce a reciprocating motion of a sleeve massaging the man's penis.

The prior art U.S. Pat. No. 5,501,650 issued to Gellert discloses an automated masturbatory device that comprises a variable speed motor powering a crankshaft driven sealed transducer producing pneumatically induced reciprocating motion of a receiver when a male organ is inserted. The invention employs a hermetic system to prevent loss of synchronization. The device receiver is designed with an inner liner compliant enough to accommodate a plurality of sizes and shapes of male penises.

Still another type of external stimulation device is disclosing U.S. Pat. No. 5,928,134 issued to Vergara. The Vergara device, which is purportedly useful for coitus performance when the erection is nonexistent or insufficient in intensity or duration, comprises a support having a rigid core and a softer lining, a fastener made of rigid material that is attached to the support by means of two hinges and a tie made of soft, flexible material that maintains the fastener well-tightened to the body by pulling from the front and rear.

As will be better understood from the discussion which follows, the novel apparatus of the present invention departs from the traditional design of the prior art external stimulation devices and provides a compact device that is of a unique, simple construction that is easy to use and one which provides intense sexual pleasure for any male user by a strategically positioned massage head disposed proximate the penile anchor of the user.

### SUMMARY OF THE INVENTION

By way of brief summary, the massage device of the present invention comprises a generally U-shaped base having spaced-apart sides interconnected by a bight portion, a transversely extending pivot rod adjustably connected to the base for movement longitudinally of the spaced-apart sides and a massage assembly pivotally connected to the pivot rod for movement between first and second positions. In one form of the invention, the massage assembly comprises a longitudinally extending support having first and second extremities and a uniquely configured massage head connected to the support proximate its first extremity. An outwardly extending hand-grip member is affixed to the support proximate its second assembly and functions to impart controlled pivotal movement of the massage assembly about the pivot rod. When the massage device is appropriately positioned between the user's legs in a manner to place the massage head in close proximity with the penile root, the pivotal movement of the massage assembly imparts an angularly up and down massaging movement to the massage head to thereby cause a pleasurable sensation to the user.

With the forgoing in mind, it is an object to the present invention to provide a novel, easy to use massaging device that enables a highly effective coupling between the penile anchor of the user and the massage head of the device so as to provide a most pleasurable and natural massaging action.

Another object of the invention is to provide a uniquely configured massage device that can be strategically positioned between the user's legs in such a manner that upon operation of the device, the massage head of the device will be moved upwardly and downwardly relative to the user's penile head so as to cause intense pleasure to the user.

Another object of the invention is to provide a massage device of the aforementioned character that is easy to use with a minimum of instruction.

Another object of the invention is to provide a massage device as described in the preceding paragraphs in which a plurality of massage heads of different shapes can be readily attached to the longitudinally extending support of the device.

Another object of the invention is to provide them the size device of the character described that is readily adjustable in a manner to precisely position the selected massage head relative to the user's penile head so that a maximum of pleasure can be achieved.

### BRIEF DESCRIPTION OF THE DRAWINGS

FIG. 1 is a generally perspective view of one form of the massage device of the present invention.

FIG. 2 is a generally perspective view of one of the massage heads that can be interconnected with the massage device shown in FIG. 1.

FIG. 3 is a generally perspective view of an alternate form of massage head that can be interconnected with them of sighs device shown in FIG. 1.

FIG. 4 is a front view of the massage device illustrated in FIG. 1.

FIG. 5 is a side-elevational view of the massage device.

FIG. 6 is an enlarged, cross-sectional view taken along lines 6-6 of FIG. 5.

FIG. 7 is an enlarged cross-sectional view taken along lines 7-7 of FIG. 5.

FIG. 8 and is a side-elevational view, similar to FIG. 5, but showing the degrees of movement of the grip member and the massage head of the device.

FIG. 9 is an enlarged cross-sectional view taken along lines 9-9 of FIG. 8.

FIG. 10 is a top plan view of one form of the massage device of the invention.

FIG. 11 is an enlarged cross-sectional view of the central portion of FIG. 10 but showing one of the massage heads connected to the support assembly of the device.

FIG. 12 is a cross-sectional view taken along lines 12-12 of FIG. 11.

FIG. 13 is a cross-sectional view taken along lines 13-13 in FIG. 12.

FIG. 14 is a cross-sectional view similar to FIG. 7, but showing the sides of the device base deformed inwardly.

### DESCRIPTION OF THE INVENTION

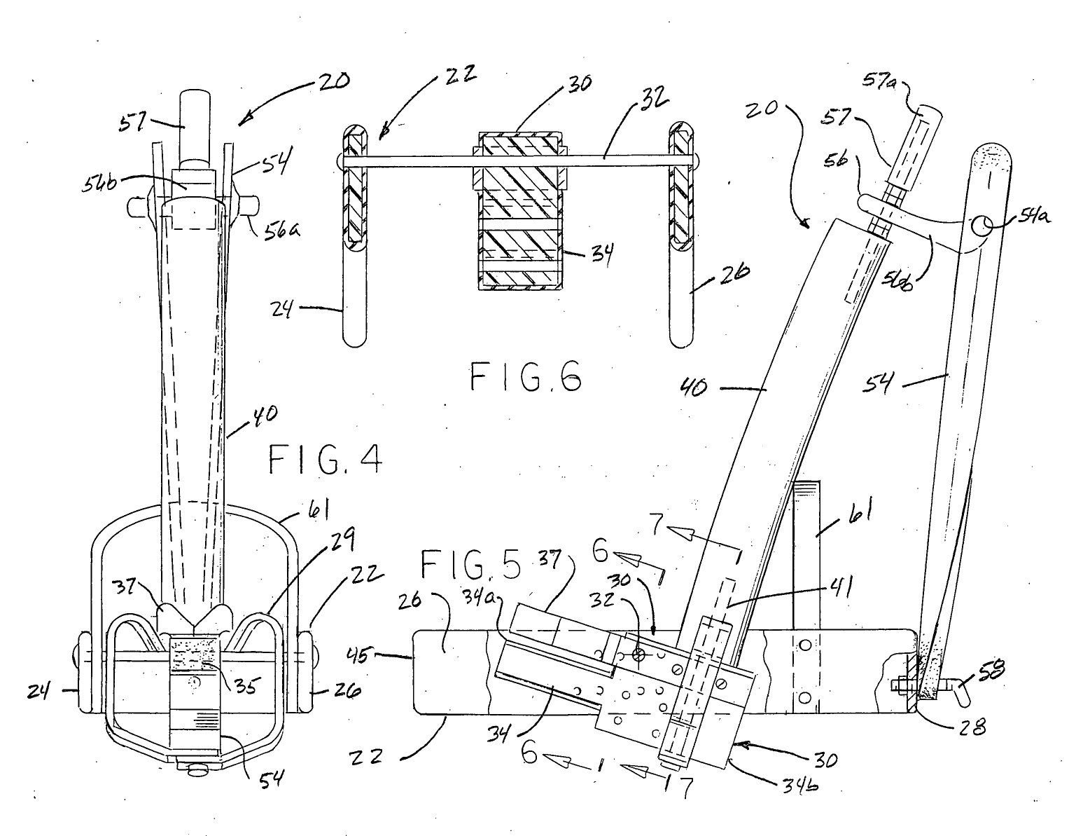

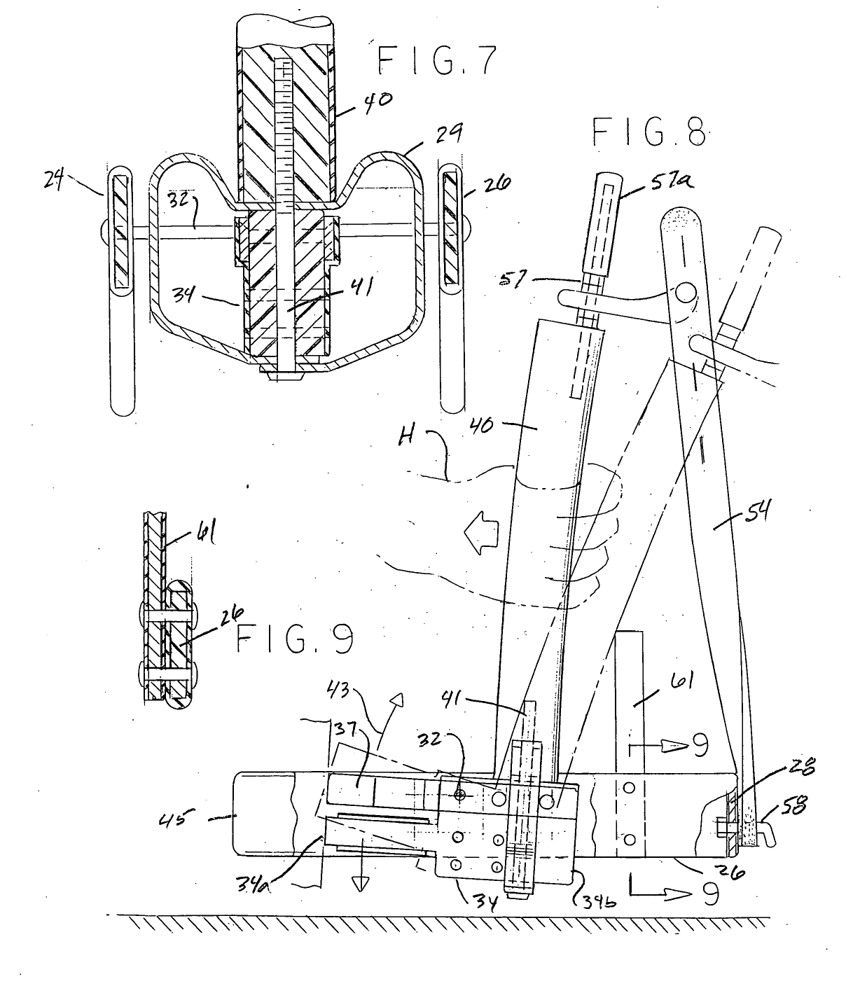

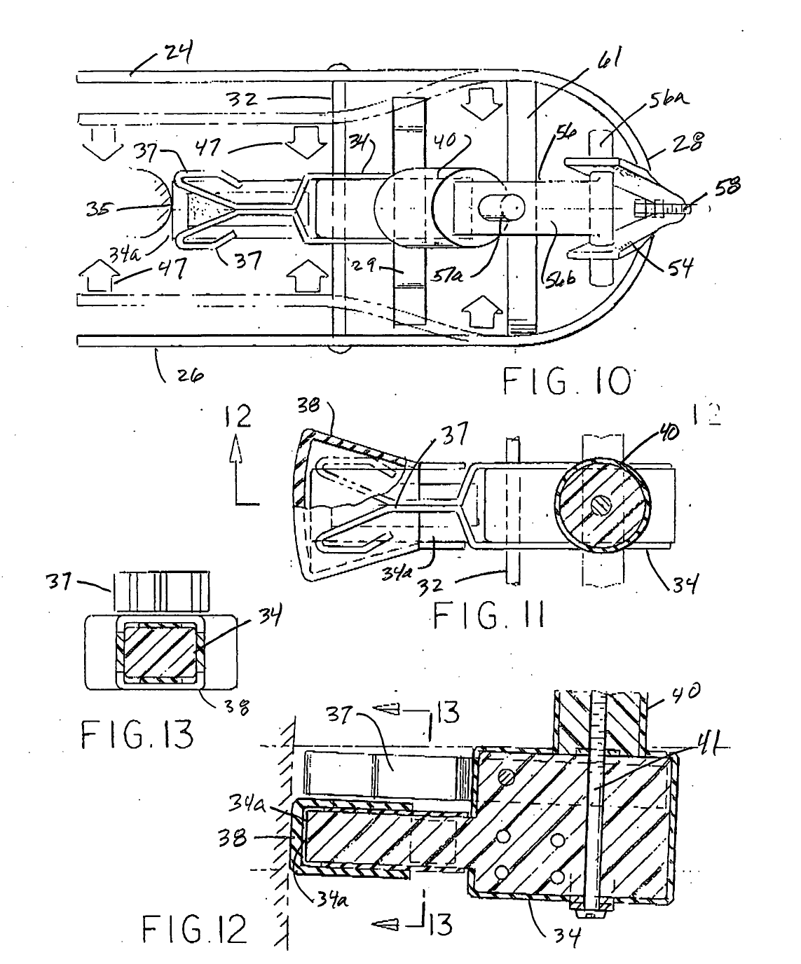

Referring to the drawings and particularly to FIGS. 1 through 5, one form of the massage device of the invention for massaging the user at a location proximate the penile root is there shown and generally designated by the numeral 20. In this embodiment of the invention, the massage device comprises a generally U-shaped base 22 having yieldably deformable, spaced-apart sides 24 and 26 that are interconnected by a bight portion 28. A massage assembly, generally designated by the numeral 30, is pivotally connected to a transversely extending pivot rod 32 that is, in turn, connected to spaced-apart sides 24 and 26. For reasons presently to be described, pivot rod 32, which spans sides 24 and 26, is adjustably connected to base 22 for movement longitudinally of spaced-apart sides 24 and 26. A yieldably deformable, spring-like spacer member 29 maintains the sides in a first spaced-apart configuration (see FIG. 7). As the sides of the base member 22 are moved inwardly in the manner shown by the phantom lines in FIG. 10, spacer member 29 is deformed into the configuration shown in FIG. 14.

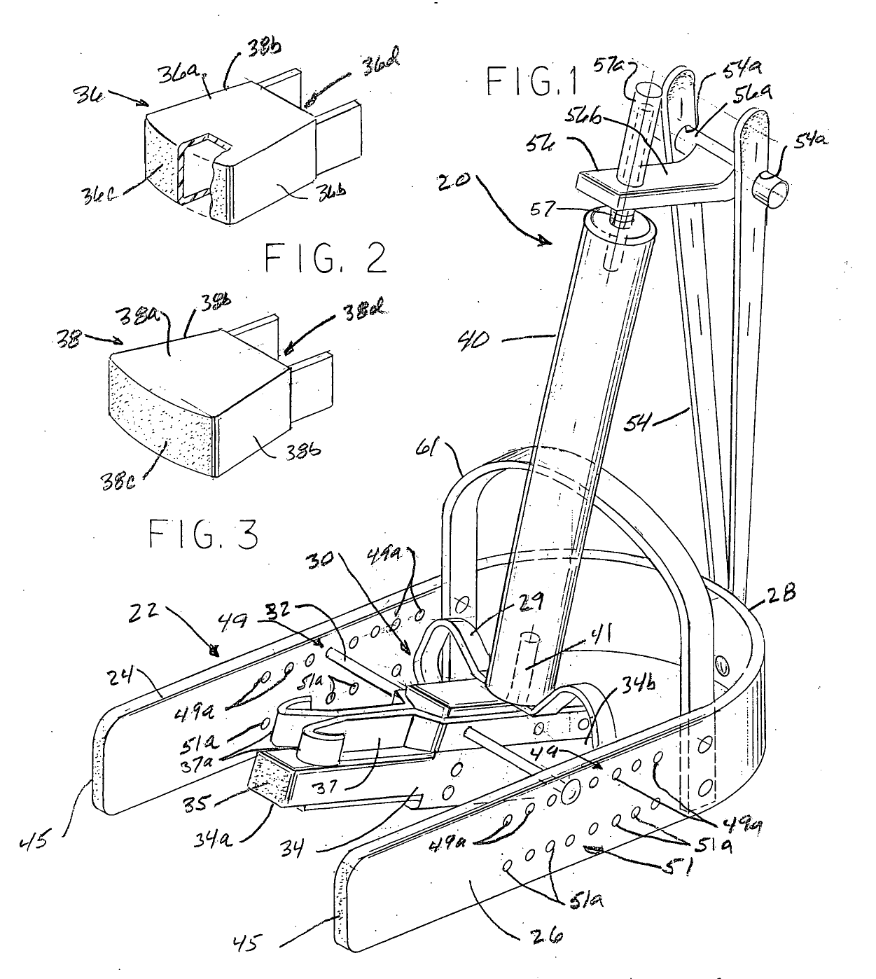

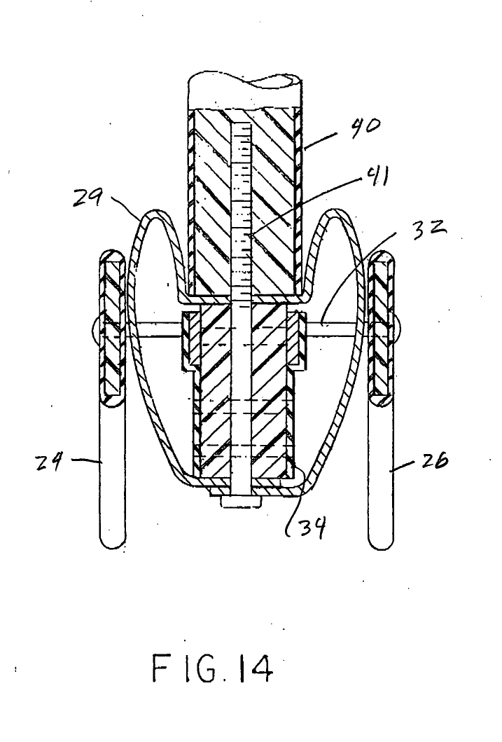

In the present form of the invention, the massage assembly 30 comprises a longitudinally extending support 34 having first and second extremities 34a and 34b. First extremity 34a is provided with a massage, or skin-stimulating surface 35 for contact with the user in a manner presently to be described. A selected one of the first and second massage heads 36 and 38, which are of the character shown in FIGS. 2 and 3 can be removably interconnected with first extremity 34a of the longitudinally extending support 34. More particularly, the massage heads include body portions 36a and 38a, tapering sides 36b and 38b and the massage surfaces 36c and 38c. The massage heads also include access openings 36d and 38d, which telescopically received first extension 34a of longitudinally extending support 34 in the manner illustrated in FIGS. 11 through 13.

Interconnected with support 34 is a secondary, yieldably deformable, generally yoke-shaped skin stimulator assembly 37 having curved stimulating extremities 37a.

As illustrated in FIGS. 1, 5 and 8, massage assembly 30 further includes an angularly, upwardly extending hand-grip member 40 that is interconnected in any suitable manner with longitudinally extending support 34 proximate its second extremity 34b. In the manner illustrated by the phantom lines in FIG. 8 of the drawings, hand grip member 40 is adapted to be gripped by the hand “H” of the user to impart pivotal movement to the massage assembly about pivot rod 32. As the hand-grip member 40 is controllably moved forwardly and rearwardly in the manner illustrated by the phantom lines of FIG. 8, the massage assembly 30 will be caused to pivot pivoted about pivot rod 32. As the massage assembly is so pivoted, extremity 34a travels angularly upwardly and downwardly as indicated by the arrows 43 of FIG. 8. This controlled upward and downward travel of extremity 34a pleasurably massages the penile root of the user when the device is positioned between the user's legs in a manner such that the end portions 45 of spaced-apart sides 24 and 26 rest against the inner thigh portions of the user and extremity 34a rests against the penile root. In this regard, it is to be noted that sides 24 and 26 of base 22, which can be moved inwardly and outwardly as indicated by the arrows 47 of FIG. 10, are provided with an upper course 49 of longitudinally spaced-apart apertures 49a. Similarly sides 24 and 26 of base 22 are provided with a lower course 51 of longitudinally spaced-apart apertures 51a that are located below spaced-apart apertures 49. With this construction it is apparent that the pivot rod 32 can be moved forwardly and rearwardly as well as upwardly and downwardly of sides 24 and 26 by positioning the extremities of the pivot rod in a selected one of the longitudinally spaced-apart apertures 49a and 51a that are formed in the spaced-apart sides. This movement of the pivot rod enables the extremity 34a of support 34 to be precisely adjustably positioned proximate the anchor, or penile root, of the user.

To ensure a smooth and well-controlled upward and downward massaging movement of extremity 34a of support 34 and of the massage heads connected thereto, biasing means are provided for yieldably resisting the pivotal movement of the massage assembly about the pivot rod between the first and second positions. This important biasing means, which can comprise any type of elastic member, such as a spring or elastic strap, is here provided in the form of an elongated, elastomeric strap 54 that extends between base 22 and a connector segment 56 that is connected to and extends rearwardly from the hand grip member 40 in the manner illustrated in FIGS. 1 and 5 of the drawings. Connector segment 56 here comprises a transversely extending rod-like portion 56a and an outwardly extending tongue like portion 56b.

As shown in FIGS. 1 and 10, elastomeric strap 54 is provided with spaced-apart apertures 54a and 54b which are configured to closely receive the opposite end portions of transversely extending rod portion 56a. Similarly, the bight portion 28 of base 22 is provided with a hook-like element 58 to which the elastomeric strap 54 is interconnected in the manner best seen in FIG. 5.

In using the apparatus of the invention, with the user lying down and bending his back forward, perhaps using a pillow to support the back, the apparatus in the configuration shown in FIG. 1 of the drawings is positioned using one hand between the user's legs in a manner such that the skin-stimulating surface 35 is in close proximity with the penile anchor. If the sides of the base 24 and 26 do not comfortably engage the inner portions of the user's legs, the sides can be yieldably deformed into the configuration shown in FIG. 10 of the drawings. In a similar manner, by repositioning the pivot rod into a selected aperture of aperture course 49a, the massage assembly 30 can be moved forwardly or rearwardly relative to sides 24 and 26 to bring the skin-stimulating surface 35 into comfortable contact with the penile root or anchor. Additionally, if desired, the massage assembly 30 can be vertically adjusted relative to sides 24 and 26 by repositioning the pivot rod into a selected one of the apertures in the upper and lower aperture courses 49a and 51a.

With the massage assembly 30 correctly positioned, the user can grip the hand-grip member 40 with his other hand and controllably move the hand-grip member forwardly and rearwardly in an oscillating motion causing be massage assembly 30 to pivot about pivot rod 32. This, of course, causes the skin-stimulating surface 35 as well as the curved stimulating extremities 37a of the secondary, yieldably deformable, generally yoke-shaped skin stimulator assembly 37 to move upwardly and downwardly relative to the penis. As previously mentioned, the elongated, elastomeric strap 54 of the apparatus functions to assist the user and accomplishing a smooth, pleasurable oscillating movement of the hand-grip member and the skin-stimulating surface. As upstanding, generally U-shaped stop member 61, which is connected to base 22 in the manner shown in FIG. 1, functions to limit the rearward travel of the hand-grip member 40 and to stop movement of the hand-grip member of at the second position.

As the penis enlarges, the user can, if desired interconnect a selected one of the massage heads 36 and 38 to extremity 34a in the manner illustrated in FIGS. 11 through 13 in order to obtain maximum pleasure. In this regard, it is to be understood that the user may initially affix a selected one of the massage heads 36 and 38 to extremity 34a if these massage heads better fit the user's anatomy.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

I claim:

1. A massage device for massaging the user at a location proximate the penile root comprising:
(a) a generally U-shaped base having spaced-apart sides interconnected by a bight portion;
(b) a transversely extending pivot rod adjustably connected to said base for movement longitudinally of said spaced-apart sides;
(c) a massage assembly pivotally connected to said pivot rod for movement between first and second positions, said massage assembly comprising:
(i) a longitudinally extending support having first and second extremities, said first extremity having a skin stimulating surface;
(ii) an outwardly extending hand-grip member for gripping by the hand of the user to impart pivotal movement to said massage assembly, said hand-grip member being connected to said support proximate said second extremity; and
(d) biasing means interconnected with said base and said hand-gripping member for yieldably resisting movement of said massage assembly between said first and second positions.

2. The massage device as defined in claim 1, further including a first massage head connected to said support proximate said first extremity.

3. The massage device as defined in claim 1, further including a generally yoke-shaped skin stimulator connected to said support proximate said first extremity.

4. The massage device as defined in claim 1, in which said biasing means comprises an elastomeric strap.

5. The massage device as defined in claim 1 in which said U-shaped base is constructed from a yieldably deformable material and in which said spaced-apart sides are inwardly deformable.

6. The massage device as defined in claim 1 in which said U-shaped base is constructed from a yieldably deformable material and in which said massage device further includes a yieldably deformable spacer member connected to said support, said spacer member being disposed intermediate said spaced-apart sides of said U-shaped base for yieldably resisting inward deformation of said spaced-apart sides.

7. The massage device as defined in claim 1 in which said outwardly extending hand-grip member is pivotally movable between first and second positions and in which said massage assembly further includes a stop member connected to said U-shaped base for stopping movement of said handle grip member at said second position.

8. The massage device as defined in claim 1 in which each of said spaced-apart sides has an upper course of longitudinally spaced-apart apertures and a lower course of longitudinally spaced apertures.

9. The massage device as defined in claim 8 in which said transversely extending pivot rod is adjustably connected to said base for vertical movement relative to said spaced-apart sides.

10. A massage device for massaging the user at a location proximate the penile root comprising:
(a) a generally U-shaped base having spaced-apart sides interconnected by a bight portion, each of said spaced-apart sides having a plurality of longitudinally spaced-apart apertures;
(b) a transversely extending pivot rod having extremities receivable within said spaced-apart apertures in said spaced-apart sides, whereby said pivot rod can be adjustably connected to said base for movement longitudinally of said spaced-apart sides;
(c) a massage assembly pivotally connected to said pivot rod for pivotal movement between first and second positions, said massage assembly comprising:
(i) a longitudinally extending support having first and second extremities, said first extremity having a skin stimulating surface;
(ii) a first massage head connected to said support proximate said first extremity; and
(iii) an outwardly extending hand-grip member for gripping by the hand of the user to impart pivotal movement to said massage assembly, said hand-grip member being connected to said support proximate said second extremity and a being pivotally movable between first and second positions; and
(d) biasing means interconnected with said base and said hand-gripping member for yieldably resisting movement of said massage assembly between said first and second positions, said biasing means comprising an elastomeric strap.

11. The massage device as defined in claim 10 in which said massage assembly further includes a generally yoke-shaped skin stimulator connected to said support proximate said first extremity.

12. The massage device as defined in claim 10 in which said massage assembly further includes a connector segment connected to and extending from said hand-gripping member, said elastomeric strap being connected to said connector segment.

13. The massage device as defined in claim 10 in which said U-shaped base is constructed from a yieldably deformable material and in which said spaced-apart sides are inwardly deformable.

14. The massage device as defined in claim 13 in which said massage device further includes a yieldably deformable spacer member connected to said support, said spacer member being disposed intermediate said spaced-apart sides of said U-shaped base for yieldably resisting inward deformation of said spaced-apart sides.

15. The massage device as defined in claim 14 in which each of said spaced-apart sides has an upper course of longitudinally spaced-apart apertures and a lower course of longitudinally spaced apertures.

16. The massage device as defined in claim 15 in which said transversely extending pivot rod is adjustably connected to said base for vertical movement relative to said spaced-apart sides.

17. A massage device for massaging the user at a location proximate the penile root comprising:
(a) a generally U-shaped base having spaced-apart sides interconnected by a bight portion, each of said spaced-apart sides having a plurality of longitudinally spaced-apart apertures;
(b) a transversely extending pivot rod having extremities receivable within said spaced-apart apertures in said spaced-apart sides, whereby said pivot rod can be adjustably connected to said base for movement longitudinally of said spaced-apart sides; (c) a massage assembly pivotally connected to said pivot rod for pivotal movement between first and second positions, said massage assembly comprising:
(i) a longitudinally extending support having first and second extremities, said first extremity having a skin stimulating surface;
(ii) a generally yoke-shaped skin stimulator connected to said support proximate said first extremity,
(iii) a first massage head connected to said support proximate said first extremity; and
(iv) an outwardly extending hand-grip member for gripping by the hand of the user to impart pivotal movement to said massage assembly, said hand-grip member being connected to said support proximate said second extremity and being pivotally movable between first and second positions; and
(d) biasing means interconnected with said base and said hand-gripping member for yieldably resisting movement of said massage assembly between said first and second positions, said biasing means comprising an elastomeric strap.

18. The massage device as defined in claim 17 in which said U-shaped base is constructed from a yieldably deformable material and in which said spaced-apart sides are inwardly deformable.

19. The massage device as defined in claim 18 in which said massage device further includes a yieldably deformable spacer member connected to said support, said spacer member being disposed intermediate said spaced-apart sides of said U-shaped base for yieldably resisting inward deformation of said spaced-apart sides.

20. The massage device as defined in claim 19 in which each of said spaced-apart sides has an upper course of longitudinally spaced-apart apertures and a lower course of longitudinally spaced apertures.
